(19) United States
(12) Patent Application Publication
(10) Pub. No.: US 2008/0260660 A1
(43) Pub. Date: Oct. 23, 2008

(54) Teeth Whitening Agents

(76) Inventors: Jurgen Engelbrecht, Elmshorn (DE); J.K. Golich, Fuerth/Odw-i (DE)

Correspondence Address: EDWARDS ANGELL PALMER & DODGE LLP, P.O. BOX 55874, BOSTON, MA, 02205, US

(21) Appl. No.: 11/793,323

(22) PCT Filed: Dec. 16, 2005
(86) PCT No.: PCT/EP2005/013587

§ 371 Date: May 28, 2008

(30) Foreign Application Priority Data
Dec. 17, 2004 (DE) 10 2004 061 646.9

(51) Int. Cl. A61K 8/34; A61Q 11/00; B01F 13/00

(52) U.S. Cl. 424/53; 366/341

(57) ABSTRACT

The invention relates to light-activated teeth whitening compositions, and to the use thereof for lightening teeth. The inventive whitening agent contains riboflavin and or the derivatives thereof, which enable the oxidant in the whitening agent to decompose more rapidly, by means of radiation energy, thus effectively accelerating the whitening action. The invention especially relates to single-constituent or dual-constituent materials that have a stable pasty form and can be optionally additionally chemically activated.

The present invention relates to light-activatable “tooth-bleaching agent” compositions and to their use in whitening teeth. The dental bleaching agent composition of the present invention comprises riboflavin and/or riboflavin derivatives that through the use of radiation energy provide for more rapid decomposition of the oxidising agent contained in the bleaching agent and are accordingly able effectively to accelerate the bleaching action. One or more constituents of the present invention can be in the form of gels or in paste form and optionally may additionally be chemically activatable.

The discoloration of teeth may be caused by the natural ageing process, the consumption of certain foodstuffs and tobacco, by diseases, by injuries, medicaments and environmental conditions or it may be hereditary. Since white or light-coloured teeth are generally perceived as being more aesthetically pleasing than dark or discoloured teeth, there has long been great interest in the development of materials and methods for whitening teeth.

Some tooth-cleaning agents, such as toothpastes, dental gels and tooth powders, contain bleaching materials that release active oxygen or hydrogen peroxide. Such bleaching agents comprise peroxides, percarbonates and perborates of alkali metals and alkaline earth metals or complex compounds containing hydrogen peroxide.

One of the bleaching materials most often used in dentistry is percarbamide, also known as urea peroxohydrate or urea hydrogen peroxide, which has also been used in dentistry as an oral antiseptic for decades. Urea itself is described in the literature as a keratinisation agent for the gums; tooth-bleaching was observed to be a side-effect when contact times were prolonged. Other bleaching agents, such as, for example, peroxyacetic acid and sodium perborate, are likewise well known in the fields of medicine, dentistry and cosmetics.

Earlier patents also disclose the use of hydrogen peroxide, percarbamide and other peroxides as bleaching agents in formulations of tooth-whitening gels, with carboxypolymethylenes and other thickeners being used for the preparation of gels. Such gels can be anhydrous or water-based.

The bleaching gels available on the market, which are also referred to worldwide by the English term “bleaching gels”, are divided into three categories, namely “Power Bleaching”, “Assisted Bleaching” and “Home Bleaching” gels. In order to save time and expense, in dental practice “Power Bleaching” is the preferred method of bleaching discoloured teeth.

U.S. Pat. No. 5,648,064 (Gaffar et al., 1995) describes a two-component toothpaste in which one constituent comprises active oxygen and the second constituent comprises a manganese coordination complex (manganese gluconate) as activator. The colorants mentioned are clearly used only for cosmetic purposes.

U.S. Pat. No. 5,098,303 (Fischer, 1992), U.S. Pat. No. 5,234,342 (Fischer, 1993), U.S. Pat. No. 5,376,006 (Fischer, 1994), U.S. Pat. No. 5,725,843 (Fischer, 1998), U.S. Pat. No. 5,746,598 (Fischer, 1998), U.S. Pat. No. 5,759,038 (Fischer, 1998), U.S. Pat. No. 5,770,105 (Fischer, 1998), U.S. Pat. No. 5,785,527 (Fischer et al., 1998), U.S. Pat. No. 5,858,332 (Fischer et al., 1999), WO Pat. No. 9,937,236 (Fischer et al., 1999), U.S. Pat. No. 5,985,249 (Fischer, 1999), U.S. Pat. No. 6,036,943 (Fischer, 2000), WO Pat. No. 0,028,953 (Fischer et al., 2000), U.S. Pat. No. 6,086,855 (Fischer, 2000) and U.S. Pat. No. 6,183,251 (Fischer, 2001) describe tooth-bleaching methods and tooth-bleaching or fluoride gels which comprise as active agent hydrogen peroxide, percarbamide, sodium perborate, benzoyl peroxide, glycerol peroxide, and as additives water, glycerol, propylene glycol, polyethylene glycol, erythritol, sorbitol, mannitol, carboxypolymethylenes, thickeners, such as xanthane, talha, tragacanth, locust bean flour, guar, ghatti, furcelleran, carrageenan, alginic acids, agar, alginates, proteins, desensitising substances, fluorides such as sodium monofluorophosphate, sodium fluoride and zinc fluoride, anti-microbial substances, such as chlorohexidine, tetracycline, cetyl pyridinium chloride, benzalkonium chloride, cetyl pyridinium bromide, methyl benzoate and propyl benzoate.

As a rule, the bleaching gels are activated by heating, by irradiation of materials that absorb radiation energy, such as 9,10-bis(phenylethynyl)-anthracene, perylene, naphtho-[2,3-a]pyrene, trans-4,4′-diphenylstilbene, 9,10-diphenylanthracene, 5,12-bis(phenylethynyl)-naphthacene, coronene, fluoranthene, carotenoids, bixin, lycopene, 7-diethylamino-4-methylcoumarin, henna and alizarin, or by addition of peroxide-decomposing agents.

EP Pat. No. 0,516,872 (Cornell, 1992) and JP Pat. No. 4,257,512 (Cornell, 1992) describes a tooth-bleaching method and a dental bleaching mixture. The latter comprises an aqueous hydrogen peroxide gel or paste and an anhydrous constituent which consists of pyrogenic silicic acid, a thickener such as cellulose ether, methyl vinyl ester, a redox indicator such as guinea green and phenolphthalein, which are intended to indicate the end of the reaction, and a light-activated catalyst such as manganese sulfate and iron sulfate.

EP Pat. 0,242,585 (Friedman, 1987) describes a peroxide solution which is activated by radiation energy in the range of from 320 to 420 nm and from 500 to 1200 nm.

EP Pat. No. 1,224,925 (Banerjee & Friedman, 2002) describes the composition of a bleaching agent which may comprise manganese chloride, manganese citrate, iron sulfate, sodium carbonate, sodium hydrogen carbonate and catalase as activators. As colorants that absorb light in the range of from 360 to 500 nm there are mentioned FD & C Red 40 or FD & C Yellow 5 and FD & C Yellow 6.

U.S. Pat. No. 6,116,900 (Ostler, 2000) describes a double-chamber mixing device and a composition comprising peroxides such as percarbamide, calcium hydrogen carbonate peroxide, sodium hydrogen carbonate peroxide and hydrogen peroxide, and salts for altering the pH value to a range of from pH 7 to pH 11, such as sodium hydroxide, calcium carbonate and calcium hydrogen carbonate. In addition, thymol blue is present as colour indicator and β-carotene as colorant for shifting the absorption of the light energy from blue to blue-green (400-550 nm). Instead of a colorant it is also possible to use an inert coloured material such as glass beads or pyrogenic silicic acid for the conversion of light energy into heat.

U.S. Pat. No. 5,645,428 (Yarborough, 1997) describes a bleaching method with peroxides and catalysts using an argon laser and a carbon dioxide laser for accelerating peroxide degradation.

U.S. Pat. No. 6,149,895 (Kutsch, 2000) mentions a bleaching agent paste in which a red colorant (Quinaldine Red and Acid Red 92 are mentioned as examples) is dissolved in a solvent and is mixed with an inert, inorganic carrier material. The resulting paste is irradiated with visible light of from 400 to 550 nm or an argon laser. On the appearance of active oxygen, the bleaching paste decolorises, so that the duration of treatment can be adjusted exactly.

U.S. Pat. No. 5,766,011 (Sibner, 1998) describes a bleaching agent which has a pH value of about from 9.5 to 10 and consists of hydrogen peroxide, an inert silicon compound, sodium hydroxide and a pigmented material. That composition, by use of an argon laser, results in a shorter contact time with the teeth.

U.S. Pat. No. 6,030,222 (Tarver, 2000) describes a colorant mixture for whitening teeth. Such a bleaching agent comprises a mixture of violet and blue colorants for absorption of light from the visible range to the violet to blue-violet range, a peroxide, a hydrophilic solvent such as water, glycerol, alcohols, polyols, ketones, aldehydes, carboxylic acids, salts of carboxylic acids and amines, a gelling agent such as polycarboxylic acids, salts of polycarboxylic acids, polysaccharides, proteins, polyalkylene oxides and pyrogenic silicic acid.

A problem of the present invention was accordingly to provide a new and improved bleaching agent composition for teeth which enables an effective bleaching action to be obtained, while the application time is short, and which can be used both as a one-component system and as a two-component system.

That problem is solved by a dental bleaching agent composition that comprises an oxidising agent (a), especially a peroxide, and riboflavin and/or riboflavin derivative(s) (b).

Preferably the oxidising agent such as the peroxide (a) is present in the form of a gel or paste, especially a gel.

Dental bleaching agents can thus be prepared more easily and more homogeneously, can be metered more accurately and can be applied more easily to the teeth being treated.

The bleaching agent composition can optionally also comprise an activator (c) which is able to activate the bleaching action of the peroxide or the peroxide gel (a). Preferably the activator is present in the form of a gel or paste, especially a gel, with the result that the preparation, metering and use of the bleaching agent is further facilitated and improved.

It is further preferable for the total bleaching agent composition to be in the form of a gel or in the form of a paste.

According to a preferred embodiment, the constituents (a) and (b) are in the form of a mixture (one-component material).

According to a further preferred embodiment, the constituents (a) and (c) are present separately from one another and are used separately from one another, while constituent (b) can be in admixture with the constituents (a) and/or (c) (two-component material).

According to the invention, the oxidising agent (a), based on the total bleaching agent composition, is used, for example, in an amount of from 1 to 90% by weight, preferably in an amount of from 2 to 70% by weight and especially in an amount of from 5 to 55% by weight (all percentages in the present invention relate to weight, and are therefore percent by weight).

It is also preferable for constituent (b), based on the total bleaching agent composition, to be used in an amount of from 0.005 to 15% by weight, preferably in an amount of from 0.01 to 10% by weight and especially from 0.05 to 5% by weight.

Furthermore, the constituent (c), based on the total bleaching agent composition, can be used, for example, in an amount of from 0.01 to 15% by weight, preferably from 0.02 to 10% by weight and especially from 0.05 to 5% by weight.

Preferably, the two constituents (a) and (c) are mixed immediately before being used for bleaching teeth.

Further preference is given to a double-chamber mixing device, with one chamber containing the peroxide gel (a) and the other chamber containing the activator gel (c), component (b) being contained in one or both chambers.

It has been found that riboflavin and also riboflavin derivatives exhibit surprisingly high effectiveness in the decomposition of the oxidising agent contained in the bleaching agent by light energy and accordingly can effectively accelerate the bleaching action.

The dental bleaching agent of this invention comprises an oxidising agent (a), especially one, two or more orally compatible oxidising agents, such as, for example, a peroxide. Examples of peroxides and/or constituents of peroxide mixtures that can be used in the preparation of the bleaching agent include hydrogen peroxide, percarbamide, sodium perborate, potassium peroxomonosulfate, potassium chlorate, potassium percarbonate, sodium percarbonate, calcium peroxide, magnesium peroxide, perphosphates, persilicates, benzoyl peroxide, glycerol peroxide, calcium hydrogen carbonate peroxide and sodium hydrogen carbonate peroxide. Hydrogen peroxide, percarbamide, sodium perborate and potassium peroxomonosulfate are preferred as bleaching agents. Hydrogen peroxide, percarbamide, sodium perborate and/or potassium peroxomonosulfate and/or mixtures of one, two or more of those compounds are preferably present in the bleaching agent composition preferably in an amount of from 5 to 70% by weight, especially in an amount of from 5 to 55% by weight. Preferably, the peroxide or peroxide gel has a pH value in the range of from 2 to 7. The further constituent (c) which may be present is an activator, especially an activator gel. The expression “activator gel” denotes compounds and/or compositions that are able to activate the bleaching action of the oxidising agent. Activator gels can be alkaline gels. They preferably comprise alkali metal and/or alkaline earth metal salts. As activators or decomposition catalysts it is also possible, however, to select salts or complexes especially from the group of copper, manganese and/or iron, more especially organometal complexes or salts such as, for example, acetylacetonates, gluconates, lactates, fumarates, naphthenic acid salts, metallocenes, oxalates, citrates, sulfates, oxides, acetates and/or mixtures thereof. In accordance with their chemical character they exhibit violent to mild reactions during the decomposition of the oxidising agents, especially the peroxides. In the case of alkaline gels, preference is given to pH values of from 8 to 11. Metal complexes and salts are preferably used in amounts of from 0.01 to 15% by weight, especially in amounts of from 0.05 to 5% by weight, based on the total bleaching agent composition.

Preferably the peroxide gel and/or the activator gel comprise a base material and/or a thickener, it being preferred when both constituents comprise a base material and/or a thickener.

As gel formers or thickeners for the preparation of the peroxide gel and the optional activator gel there can be used, for example: cellulose polymers, polycarboxylic acids, pyrogenic silicon dioxide, poly(meth)acrylic acids, polysaccharides, polyvinyl butyrals, alginates, coumarone resins, shellac, xanthane, tragacanth, guar, carrageenan, alginic acids etc. and/or mixtures thereof. They can be present in the constituents, based on the total bleaching agent composition, in an amount of from 0.01 to 20% by weight, preferably in an amount of from 0.05 to 15% by weight.

As base material for the preparation of stable gels, water or water in combination with other compounds or mixtures is often used. Such compounds or mixtures comprise polyols such as polyethylene glycol, sorbitol, polypropylene glycol, propylene glycol, glycerol, ethanol, acetone, ether, acetates, xylitol and others and/or mixtures thereof. Polyols such as glycerol and/or propylene glycol and/or demineralised water are preferred in this invention. They are present either alone or as mixtures in an amount, based on the total bleaching agent composition, of from 0.1 to 98% by weight, and preferably in an amount of from 0.5 to 95% by weight.

Furthermore, the bleaching agent composition of the present invention comprises riboflavin and/or riboflavin derivative(s). That constituent (b) can be added to the oxidising agent (a) and/or the activator (c). By means of constituent (b), light energy is converted into heat and accordingly the bleaching action of the dental bleaching agent composition is improved and accelerated. For the action it is not important in which constituent the riboflavin and/or the riboflavin derivatives is/are present. It is preferable, however, to select the constituent in which it is most stable. The amount of riboflavin and/or riboflavin derivatives is from 0.001 to 15% by weight, preferably from 0.005 to 10% by weight.

Examples of riboflavin derivatives are: riboflavin-5′-phosphate, riboflavin-5′-malonate; preferred riboflavin derivatives are riboflavin-5′-phosphate and riboflavin-5′-malonate.

The compositions of the present invention can comprise further constituents, such as, for example, stabilisers such as alkali metal polyphosphates, alkali metal pyrophosphates, ethylene diamine tetraacetic acid and salts thereof, tartaric acid and salts thereof, citric acid and salts thereof, gluconic acid and salts thereof, triethanolamine, tin nitrate, adipic acid, tin phosphate, succinic acid etc., such as, for example, constituents that alter the pH value such as alkali metal and alkaline earth metal salts, such as, for example, vitamins as anti-inflammatories, as well as flavourings such as, for example, peppermint, vanilla etc., colorants for colouring purposes and as indicators, preservatives, fluoride derivatives, wetting agents etc. They can be present in the gels of this invention both on their own and in mixtures.

### EXAMPLES

In order to demonstrate the surprisingly high level of effectiveness of riboflavin and/or riboflavin derivatives, the rise in temperature during irradiation was measured. The dental photocuring apparatus used was the PowerPac light apparatus from American Dental Technologies. The rises in temperature measured during the irradiation are listed in Table 1.

### One-Component Gels

Glycerol was used as base material and pyrogenic silicon dioxide as thickener.

| Comparison | | | |
|---|---|---|---|
| Example 1: | glycerol | 73.00% | w/w |
| | thickener | 5.00% | w/w |
| | percarbamide | 22.00% | w/w |
| Example 2: | glycerol | 73.00% | w/w |
| | thickener | 4.99% | w/w |
| | percarbamide | 22.00% | w/w |
| | riboflavin-5′-phosphate | 0.01% | w/w |
| Example 3: | glycerol | 83.00% | w/w |
| | thickener | 5.99% | w/w |
| | percarbamide | 11.00% | w/w |
| | riboflavin-5′-phosphate | 0.01% | w/w |

### Two-Component Gels

Glycerol was used as base material and pyrogenic silicon dioxide as thickener.

| Comparison | | | | |
|---|---|---|---|---|
| Example 4: | a) | glycerol | 83.00% | w/w |
| | | thickener | 6.00% | w/w |
| | | percarbamide | 11.00% | w/w |
| | b) | glycerol | 93.00% | w/w |
| | | thickener | 6.40% | w/w |
| | | Fe(II) sulfate | 0.60% | w/w |
| Example 5: | a) | glycerol | 83.00% | w/w |
| | | thickener | 5.99% | w/w |
| | | percarbamide | 11.00% | w/w |
| | | riboflavin-5′-phosphate | 0.01% | w/w |
| | b) | glycerol | 93.00% | w/w |
| | | thickener | 6.40% | w/w |
| | | Fe(II) sulfate | 0.60% | w/w |
| Example 6: | a) | glycerol | 73.00% | w/w |
| | | thickener | 4.99% | w/w |
| | | percarbamide | 22.00% | w/w |
| | | riboflavin-5′-phosphate | 0.01% | w/w |
| | b) | glycerol | 93.00% | w/w |
| | | thickener | 6.40% | w/w |
| | | Fe(II) sulfate | 0.60% | w/w |

### Results:

TABLE 1
Rise in temperature of one-component and two-component bleaching
material on irradiation with the PowerPac apparatus

| | |
|---|---|
| | PowerPac |
| Bleaching materials | ΔT |

One-component gels

| | |
|---|---|
| Comparison Example 1 | c. 6.9° C. |
| Example 2 | c. 10.0° C. |
| Example 3 | c. 11.7° C. |

Two-component gels

| | |
|---|---|
| Comparison Example 4a) + 4b) | c. 6.7° C. |
| Example 5a) + 5b) | c. 8.5° C. |
| Example 6a) + 6b) | c. 8.8° C. |
| QuasarBrite ™ | c. 4.2° C. |
| (Comparison Example 7) | |

The irradiation times for (Comparison) Examples 1 to 6 and QuasarBrite™ (a light-activated “bleach” product as comparison, which contains Thermal Absorption Crystals™ for light activation) were 30 seconds with the PowerPac apparatus. (Comparison) Examples 4 to 6 were irradiated shortly after the chemical reaction of the two-component material. In the last-mentioned Examples, commercially available 1:1 double-chamber syringes (MixPac, Switzerland) were used, so that the actual peroxide content is only half the amounts in the Examples. Table 1 therefore shows the superior effectiveness of the riboflavin derivative used.

1-21. (canceled)

22. A dental bleaching agent composition comprising (a) an oxidising agent and (b) riboflavin and/or a riboflavin derivative.

23. A bleaching agent composition of claim 22 wherein the oxidising agent (a) is a peroxide.

24. A bleaching agent composition of claim 22 wherein the composition further comprises (c) an activator.

25. A bleaching agent composition of claim 22 wherein at least one of the constituents (a) and (c) is in the form of a paste or gel.

26. A bleaching agent composition of claim 22 wherein the composition is in the form of a gel or paste.

27. A bleaching agent composition of claim 25 wherein constituent (a) has a pH of from 2 to 7.

28. A bleaching agent composition of claim 23 wherein the peroxide (a) is hydrogen peroxide, percarbamide, sodium perborate or potassium peroxomonosulfate or a mixture thereof.

29. A bleaching agent composition of claim 23 wherein constituent (a) is contained in an amount of from 2 to 90% by weight, based on the total bleaching agent composition.

30. A bleaching agent composition of claim 24 wherein the activator (c) comprises at least one alkaline additive.

31. A bleaching agent composition of claim 30 wherein the alkaline additive comprises or consists of an alkali metal salt or an alkaline earth metal salt.

32. A bleaching agent composition of claim 24 wherein the activator (c) comprises at least one salt or at least one complex of a metal from the group copper, manganese and iron.

33. A bleaching agent composition of claim 31 wherein the activator (c) is an acetyl-acetonate, gluconate, lactate, fumarate, naphthenic acid salt, metallocene, oxalate, citrate, sulfate, oxide, acetate or a mixture thereof.

34. A bleaching agent composition of claim 32 wherein the activator (c) is contained in an amount of from 0.01 to 15% by weight, based on the total bleaching agent composition.

35. A bleaching agent composition of claim 32 wherein the activator (c) is contained in an amount of from 0.05 to 5% by weight, based on the total bleaching agent composition.

36. A bleaching agent composition of claim 22 wherein the riboflavin derivative (b) is riboflavin-5′-phosphate or riboflavin-5′-malonate.

37. A bleaching agent composition of claim 22 wherein constituent (b) is contained in an amount of from 0.005 to 15% by weight, based on the total bleaching agent composition.

38. A bleaching agent composition of claim 25 wherein constituent (b) is present in the peroxide gel (a) or in the activator gel (c) or in both.

39. A kit comprising a bleaching agent composition of claim 24 wherein constituent (a) is present separately from constituent (c).

40. A double-chamber mixing device having a static mixer, comprising a bleaching agent composition of 24, wherein a first chamber of the device comprises constituent (a), a second chamber of the device comprises constituent (c) and at least one of the first and second chambers comprise constituent (b).

41. A method for whitening teeth of subject comprising:
applying a bleaching agent bleaching agent composition of claim 22 to teeth of the subject.
